(19) United States
(12) Patent Application Publication
(10) Pub. No.: US 2005/0075543 A1
(43) Pub. Date: Apr. 7, 2005

(54) Method of anonymous medical testing and providing the patient with the test results

(76) Inventor: Charles A. Calabrese, St. James, NY (US)

Correspondence Address: TRASK BRITT, P.O. BOX 2550, SALT LAKE CITY, UT, 84110, US

(21) Appl. No.: 10/679,016

(22) Filed: Oct. 3, 2003

(51) Int. Cl. G06F 17/60; A61B 5/00

(52) U.S. Cl. 600/300; 705/2

(57) ABSTRACT

A system for delivering confidential test results to anonymous subjects is described. The system includes procuring a unique personal identifier or personal identification number (PIN) from a first source, which communicates that PIN to a second source that is capable of taking and/or testing a suitable test specimen from the patient being tested. The second source receives a biological sample identified by the PIN from the patient or a patient representative and performs a test on the biological sample to detect genetic and/or medical information and provides a test result. The test result is then provided to the patient through an internet site, phone system or in person, and accessed by the patient through the unique PIN.

### TECHNICAL FIELD

The invention relates to maintaining the anonymity of a subject while providing sensitive information, such as genetic or medical test results, to the subject.

### BACKGROUND

Two random people have approximately 99.9% identical genetic material. That 0.1% difference not only distinguishes us from one another, but can serve as a basis for excluding individuals from insurance, employment, adoption, education, and numerous other life events. For example, a federal study of genetic discrimination based on genotype included treated hereditary hemochromatosis (HH) as one of only five genotypes possibly providing a basis for discrimination. HH was selected because if diagnosed early and properly managed, the affected individual has no greater morbidity or mortality risk than a member of the general population. Therefore, the sole source of discrimination would be based on genotype, not phenotype. Since the publication of those results, state and federal legislatures have, to varying degrees, attempted to legislate protection from such discrimination. Similar reports have indicated potential consequences, including loss of medical insurance that can be suffered, if a subject tests positive for a particular disease or genetic pre-disposition. Beyond the negative effects that can be suffered by the patient or subject, longer term negative effects, such as the use of family history information in setting insurance premiums, may adversely affect a potential patient's children and thereby discourage the patient from having a test performed (Human Genetics Commission (2001), Comments to inform the Government response to the House of Commons report on Genetics and Insurance, HC 174).

Many people are reluctant to be tested for diseases or genetic pre-dispositions, such as HIV, herpes, or Huntington's disease. Not only do people have to deal with the emotional ramifications of this highly sensitive information, but they also have to deal with the potential that such information may be discovered and thereafter used to their detriment. This discrimination can result in loss of employment, loss of insurance, loss of housing, and/or loss of friendships and family ties.

Genetic tests are currently available for more than 400 diseases, with tests for hundreds more under development. The number of available genetic tests is expected to increase dramatically as a result of the completion of the Human Genome Project. More than 175,000 genetic tests were performed in 1996 alone and the number has been increasing approximately 30% per year. The true benefit of genetic testing or testing for infections will be more fully realized if the negative consequences of a positive test result can be more effectively managed.

AIDS and herpes are examples of serious modern-day viral health issues that do not have known cures. Early detection and treatment provides the best chance for prolonging the subject's life and preventing the spread of the virus itself. However, individuals are reluctant to submit to testing because, among other things, they fear that a positive test result will not be kept confidential and may have severe negative effects.

Although early detection of infection by HIV is vitally important, only about 8% of adult Americans are tested annually. It is currently estimated that the number of at-risk individuals being tested for the HIV virus would increase to approximately 29% if a diagnostic procedure was available and the individual's confidentiality could be maintained. However, getting tested may subject a person testing positive for the HIV virus to being ostracized from their community, labeled a drug addict or homosexual, dropped by their insurance provider, and/or fired from their job. A positive diagnosis for the HIV virus may lead to these results even though the subject may have contracted the virus through a blood transfusion. Taking into account these factors, the decision to test may be a difficult choice to make. However, there are good reasons to test, including, but not limited to, a need to know, anxiety relief, protecting oneself, protecting one's sexual partners, protecting one's children, and obtaining early medical intervention. If a patient can obtain test results without compromising their anonymity, the risk of negative effects is lowered, thereby increasing the chance that the patient will have the test performed.

The lack of testing may have serious repercussions for both the patient and society in general. Late diagnosis of serious health issues may decrease treatment options and increased cost for those treatments. For example, a test showing a predisposition to breast cancer (e.g., BRAC 1 or BRAC2 tests) may lead to increased screening and an earlier diagnosis. An earlier diagnosis increases the chance that cancer can ultimately be treated and may reduce the cost of treatment. However, a positive test result for a mutation in BRAC 1 or BRAC 2 does not indicate the presence of breast cancer or indicate that breast cancer will develop. Thus, a woman and her female offspring have to face the risk of losing health care coverage, being fired or other negative effects, simply to detect an increase in the woman's probability of developing the disease.

The ability of the person taking the test to remain completely anonymous remains a serious problem. In the past, systems have been developed that attempt to protect individuals from the general dissemination of test results. Medical practices and policies, like the doctor-patient privilege, attempt to shield people from embarrassing exposure and public ridicule. There are clinics which have procedures for anonymous testing. These clinics use numeric identifiers to protect patient confidentiality. However, the patient must appear at the clinic and provide personal information to the same clinic that assigns the numeric identifier. Accordingly, complete anonymity may not be maintained.

Tests have been developed and marketed that allow people to take a blood sample at home and mail it to a medical laboratory for analysis. However, these tests may still require the person taking the test to give the laboratory his or her name and address. The loss of anonymity thereby discourages people from taking the test. Furthermore, the previous personal testing kits have typically required the user to supply one small blood sample. While this is often sufficient for an accurate test result, it is insufficient when multiple tests are needed.

Accordingly, a medical testing system is needed that provides complete confidentiality and allows the user to remain completely anonymous. The test preferably supplies the testing facility with enough of the test specimen to ensure that the test result is as accurate as possible.

### SUMMARY OF THE INVENTION

The invention relates to a system for delivering confidential test results and counseling to numerous anonymous subjects. The methodology of the present invention involves procuring a personal identification number (PIN) from a first source, which communicates that PIN to a second, separate source that is capable of taking and/or testing a suitable test specimen from the person being tested or other unrelated entity.

One aspect of the invention relates to a method for anonymously testing a patient by assigning a unique numerical identifier to a patient and at least one test for a medical and/or genetic condition. The unique numerical identifier identifies the patient and the test to be performed for the patient. The unique numerical identifier is transmitted from a first source to a testing facility. A biological sample is received from the patient at the testing facility, wherein the biological sample is identified by the unique numerical identifier. A test is performed on the biological sample to detect a medical condition and to determine a test result. The test result is correlated to the unique numerical identifier. A request for the test result is then received from the patient, the test being identified by the unique numerical identifier. The test results are then provided to the patient, thereby protecting the identity of the patient.

Another aspect of the invention relates to a method wherein the biological sample is obtained at the testing facility and the testing facility uses the unique numerical identifier to identify the patient. In addition, the request from the patient may be conducted through an internet site and/or the test results are provided to the patient through an internet site.

Another aspect of the invention relates to a method for anonymously testing a patient, the method comprising: assigning a patient a unique numerical identifier and a test for a medical condition, wherein the unique numerical identifier identifies the patient and the test to be performed for the patient; transmitting the unique numerical identifier to a testing facility; receiving a biological sample from the patient at the testing facility, wherein the biological sample is identified by the unique numerical identifier; performing a test on the biological sample to detect a medical condition and determining a test result; correlating the test result from the test with the unique numerical identifier; receiving a request for the test result from the patient, wherein the patient is identified by the unique numerical identifier; verifying the unique numerical identifier against a database of valid unique numerical identifiers after receiving the request; and providing the test results to the patient if the unique numerical identifier is valid.

Another aspect of the invention relates to determining if the patient has a positive test result for a human malady or predisposition to a human malady, and providing or making available information regarding the human malady or the predisposition to a human malady to the patient prior to providing the test result. In addition, the invention relates to querying the patient to determine if the patient desires counseling; and providing the patient with counseling, wherein the counselor provides the test results. Alternatively, the invention relates to querying the patient regarding desired online counseling; and providing an instant messaging account number and instructions for using the instant messaging account, wherein the patient has indicated a desire for online counseling, and connecting a counselor by the instant messaging account to the patient, thereby providing additional information to the patient.

Yet another aspect of the invention relates to a system for delivering test results, which can be used in conjunction with a confidential testing kit, such as a home blood sample kit.

The invention also relates to an internet routing and handling system for delivering the test results and counseling, while maintaining the subject's anonymity. Alternatively, a telephone call routing and handling system may be used to maintain the subject's anonymity. In yet another embodiment, the invention allows the subject to meet with the service provider in person, since the service provider has no access to the subject's personal information and therefore, cannot disclose or obtain information about the patient's identity.

### BRIEF DESCRIPTION OF THE DRAWINGS

FIG. 1 shows a flow chart of the general organization of an embodiment of the invention.

FIG. 2 shows a flow chart of the general organization of the identification generation center.

FIG. 3 shows a flow chart of the general organization of the testing facility.

### DETAILED DESCRIPTION OF THE INVENTION

The present invention relates to a confidential test system for determining whether an individual may have a disease or infection (e.g., HIV virus), be predisposed to a disease, or have a particular form of a disease. An individual uses the system by purchasing an anonymous identification number through a first company or person. The first company receives the subject's payment for the desired tests. The subject is assigned a unique identification number, which may include alphabetical characters, that is then used as the sole identifier for the subject. The subject is then provided with a test kit and the location to send the sample to, or is alternatively given the address of one or more service provider(s) to choose from. The subject then provides a sample, either using the test kit at home or at the appropriate service provider. However, the subject is identified only by the unique identification number for all subsequent transactions, which include providing the sample and receiving the test results. Anonymity is further protected, since the fees to be charged by the service provider or the testing facility are paid by the first company when the identification number is transmitted to the testing facility. Thus, the testing facility has no information regarding the subject, other than the identification number. Several days later, the subject can access the results online, call a central phone number or personally visit the testing facility to obtain his or her test results. In a particular embodiment, the subject may also request or receive any necessary counseling.

Representative tests that may be used in relation to the current system and methods include, but are not limited to, Alzheimers, Osteoporosis, Drug Use, DNA Genetics, Infertility, Paternity, HIV/Aids, Hepatitis, Body Fat Scales, Breathalyzers, CardioChek, Cholesterol, Diabetes, Sexually Transmitted Diseases (STDs), Carpal Tunnel and Cancers, such as, Breast, Prostate, Colon and Skin cancer. The test may be based on saliva, hair, blood, urine, stool, or other biological samples or fluids. These tests may utilize assays that include, but are not limited to, Enzyme Linked Immunosorbent Assay (ELISA), Western Blot tests, hybridization, PCR, GC/MS, TLC and other nucleic acid and protein based tests.

Various genetic tests can be used with the present invention. These include, but are not limited to, tests for breast cancer, Haemophilia, Duchenne's muscular dystrophy, Becker's muscular dystrophy, Adrenoleukodystrophy, Beta thalassemia, Cystic Fibrosis, Fragile X, Familial Adenomatous Polyposis (FAP), Huntington's disease, Hurler syndrome, Neurofibromatosis, Phenylketonuria, Long QT syndrome, Lesch Nyhan syndrome, Spinal Muscular Atrophy, Von Hippel Lindau, hearing impairment (for example, the GJB2 gene) and Tuberous sclerosis. For a more complete list of heritable human maladies, see Candidate Genes to Inherited Diseases, available at http)://www.bork.embl-heidelberg.de/g2d, the contents of which are incorporated hereto by reference.

Any HIV, sexually transmitted disease (STD), or genetic testing system should have at least one mechanism to maintain the anonymity of the patient. In addition, the system may also include at least one mechanism to monitor: (i) if the test results have been viewed; and (ii) if the subject requires counseling and/or has received counseling. The testing system must be able to deliver confidential test results (and, optionally, counseling) to numerous subjects without the loss of the patients anonymity. Test results may be delivered over the internet, over the telephone, or in person. However, in each case, the subject's identity is maintained in confidence through the use of a unique personal numerical identifier.

The invention maintains the anonymity of the patient by separating the patient's personal information from the testing procedure. Thus, the testing is conducted in an environment where the patient is identified solely by a unique personal numerical identifier, such as a PIN. For example, the patient contacts an identification generation center that can record any necessary personal information, takes payment for the tests to be conducted, provides the patient with the necessary information regarding where and how to conduct the test, where and how to retrieve the results, and assigns the patient a personal identification number. The patient then conducts the test at home and mails the sample to an appropriate testing facility or has the sample drawn at a testing facility. However, in either case, the patient is identified at the testing facility solely by his or her PIN and not by personal information. Thus, if the patient has the sample drawn at the testing facility, the patient is admitted to the facility, where appropriate, and the sample is drawn under the patient's PIN number. As a part of maintaining the anonymity of the patient, payment for the test to be performed is, likewise, submitted to the testing facility by reference to the PIN. Thus, any personal information, such as that associated with a credit card or personal check, is not associated with the generation of the PIN and are separated from the test results. For example, transmittal of the medical test to be conducted and payment for performing and reporting the test may be transmitted by the identification-generation center (IGC) to the testing facility. Thus, the testing facility receives payment for specified tests and a PIN assigned to those tests, without compromising the patients identity. In particular, the IGC does not receive the test results. The patient's personal information, such as name or address, is thereby disconnected from the test results. Thus, test results are connected only to the PIN and are not be linked to the patient's personal information.

The invention provides a unidirectional information flow of specific information or material between an identification generation center and a testing facility. The identification generation center communicates to the testing facility a PIN and payment, wherein the payment does not provide information relative to the patient. The testing facility receives payment and a PIN, performs the tests, and makes the test results available to the patient through the use of the PIN.

In a particular embodiment, the invention provides a method of separating the patient's personal information from the test results. Specifically, the identification generation center (IGC), which has access to the patient's personal information and PIN, is separated from the testing facility, which has access to the test results and PIN. However, the IGC and testing facility are prevented from accessing information particular information from the other. Thus, the IGC does not have access to test results and the testing facility does not have access to personal information that could compromise the patient's anonymity. The invention thereby reduces the risk or prevents release of the patient's identity, for example, through mistaken or accidental release.

Test results may be released to the holder of the appropriate PIN through an anonymous telephone system, the internet (e.g., a secure web site), or in person. For example, a telephone system may be established to handle incoming telephone calls initiated by anonymous callers, each of the anonymous callers having previously provided a specimen for evaluation to a testing facility. In this example, a telephone call initiated by an anonymous caller is received by a call routing and handling system. The anonymous caller is prompted by the call routing and handling system to transmit a personal identification number (PIN) corresponding to a specimen previously provided to the testing facility by the anonymous caller. Next, the PIN transmitted by the anonymous caller is received by the call routing and handling system, and test result information is retrieved through access from a previously-received PIN submission and verification. A call handler for processing the telephone call may be selected in response to the test result information. The selected call handler may be chosen from a plurality of candidate call handlers. The telephone call is then routed to the selected call handler and test result information corresponding to the PIN is provided to the anonymous caller by the selected call handler.

The test may include a kit having all the necessary elements for the person taking the test to be able to obtain a test specimen in private and information regarding submission of the specimen in connection with the PIN. The test results and PIN may be linked by creating or activating a personal electronic file through a software program in a computer system that can be both created and subsequently accessed anonymously and remotely from a telephone or through the internet. The electronic file that is set up is identified only by the PIN. Because the file connecting the results to the PIN is created at a testing facility separate from the IGC, the identity of the person taking the test is unknown to others, thus, protecting the anonymity of the person taking the test.

In a particular embodiment, pre-test counseling regarding the test and the test results may optionally be offered by the identification generation center setting up the PIN. The pre-test counseling is an opportunity for the person taking the test to seek assistance in the form of counseling to cope with the possible results that could result if the testing reveals a positive result. Pre-test counseling may provide the patient with information regarding the appropriate tests for a particular situation and the nature of those tests. For example, the patient may seek information regarding the possibility of transmitting a medical condition, or a predisposition to a medical condition, to the patient's offspring or of developing a medical condition in the future. Pre-test counseling is counseling done prior to a test result. Therefore, a patient is unlikely to require the anonymity of the invention at that time. However, once the patient determines the appropriate test to take and receives counseling regarding the nature of the test and the consequences of a positive, negative or non-determinative result, the patient may desire to obtain the test results without disclosing their identity. Thus, the patient may seek to use the invention to preserve their identity.

When the patient taking the test anonymously retrieves the results using the PIN, the patient may also seek counseling at that time. However, once the test result is known, the patient's identity is preserved by referencing the test results through the use of the PIN. Therefore, any counseling conducted after the test results are obtained is accomplished through the use of the PIN.

In one embodiment, an IGC creates an order request form, which authorizes the testing facility to perform the testing and analyze the results for a specimen identified by the PIN associated with the specimen. The authorization may include any necessary medical consent forms, such as an informed consent form. The informed consent form typically contains a series of statements that the person being tested must read, understand, and acknowledge before a laboratory can perform any test on a specimen. Thus, it is preferable to have the informed consent form signed by the patient while in the IGC, even more preferably two consent forms are used, wherein the first form is signed by the patient and the second form references the patient by PIN only and is sent to the testing laboratory. Therefore, where informed consent forms are used, the anonymity of the patient is maintained by using the PIN to identify the patient when such documentation is supplied to the testing facility.

The test specimen is often a blood sample but can also be another body fluid or tissue like saliva, urine, feces, hair follicles or skin cells. The test specimen is then submitted for analysis at the testing facility. The test specimen is associated with the unique personal identification number to identify and preserve the anonymity of the person taking the test. If the test specimen is to be mailed to the testing facility, the sample is preferably mailed in a pre-addressed envelop without the senders return address.

Upon receipt of the test specimen, the test facility matches the specimen with the PIN. Once the analysis is completed, the results are connected to an electronic file identified by the PIN that corresponds to the PIN of the test specimen.

The person seeking the test results may then access the electronic file by calling a telephone number and entering the PIN or by logging onto a web site using the PIN. Thus, the test results are accessible only with a PIN, thereby maintaining absolute anonymity of the person having the test performed.

The electronic file is preferably configured to direct the person to the appropriate source to receive the results. For example, positive results indicating a human malady are connected to a source of counseling. The counseling may be provided by a professional counselor who does not know the identity of the person or may be electronically transmitted (e.g., a web page or a telephone message having the appropriate information for the patients condition). Alternatively, the person taking/seeking the results of the test may be encouraged to speak to a counselor, if needed.

If the test results are negative for the presence of a human malady, the patient may simply be informed of the results. However, additional information may also be provided to the patient regarding, for example, further testing. If the test results are inconclusive, the patient may be routed to a separate source of information where the reasons for the inconclusive result may be explained (e.g., insufficient specimen was supplied) and suggesting that the patient repeat the process and correct the sample error.

The test system permits anonymous testing for a particular human malady, and may include a kit having the necessary equipment for producing a suitable specimen from a person being tested, which is capable of being transported from one location to another location for analysis. Such a kit allows the specimen to be collected in private. To maintain anonymity, a PIN identifies both the person being tested and the test specimen. An electronic file is created and can be accessed by the person being tested through use of the PIN code. The electronic file includes the results of the analysis. The test results may be removed or deleted by the patient. In a particular embodiment, the patient can be provided the option of expunging selected results. The patient can opt to expunge the results or keep the results in the electronic file for future reference.

When the body fluid to be tested is blood, the kit may include all suitable equipment for blood extraction, including, for example, needles, collection containers (e.g., tubes), alcohol swabs, sterile gauzes, lancets for puncturing the skin. Such a test kit may further include at least one bandage to protect the puncture site after the blood sample is produced. The test kit (as well as other test kits described herein) may further include an appropriate mailing container, wherein the mailing container is preferably pre-addressed. The blood sample is acquired by placing collected blood on a blood specimen collection devise provided in the kit. Preferably, sufficient sample is provided to conduct the test at least twice, and more preferably, sufficient sample is provided to allow for multiple tests.

When the body fluid to be analyzed or tested is urine or a stool sample, the kit is preferably equipped with a suitable cleaning device, such as a sanitary wipe, to clean the patient and the exterior of the sample container, and at least one sample container having a securely sealable lid. Instructions regarding proper collection of the sample are preferably included with the kit. The urine or stool sample is collected by collecting enough of the specimen in the sample container provided in the kit.

When the sample to be analyzed or tested is a nucleic acid based test, the specimen may be blood, a mucosal swab, hair folicals or skin cells. Depending on the assay to be conducted more or less nucleic acid may be required and the sample container will be appropriately sized, depending on the source from which nucleic acid is to be obtained and whether amplification is possible. Where a mucosal swab is appropriate and desired, the test kit will preferably include an appropriate swab, preferably separately packaged to be unsealed immediately prior to use, and a sample contained suitable to hold the swab and maintain the sample is a satisfactory condition. The mucosal swab may be obtained by inserting a swab into the mouth and scraping the side of the check with the swab. Preferably, sufficient sample is provided to conduct the test at least twice, more preferably sufficient sample is provided to allow for additional tests. For example, multiple swabs may be provided and sent to the testing facility. Other samples and collection devises are known in the art and may be used to collect and transmit the appropriate sample.

Preferably, a testing facility should receive a sufficient quantity of a sample to perform the desired test at least twice, thereby allowing for verification of the test accuracy. The invention also provides for a testing facility to receive sufficient sample to perform additional tests, thus, the testing facility may maintain the sample and identify the same in relation with the PIN. The patient may then contact, the IGC or the testing facility to order additional tests to be performed on the sample. Preferably, where additional payments for supplemental tests are required, the patient can make those payments to the IGC in order to preserve patient anonymity. The IGC would then forward a suitable payment to the testing facility.

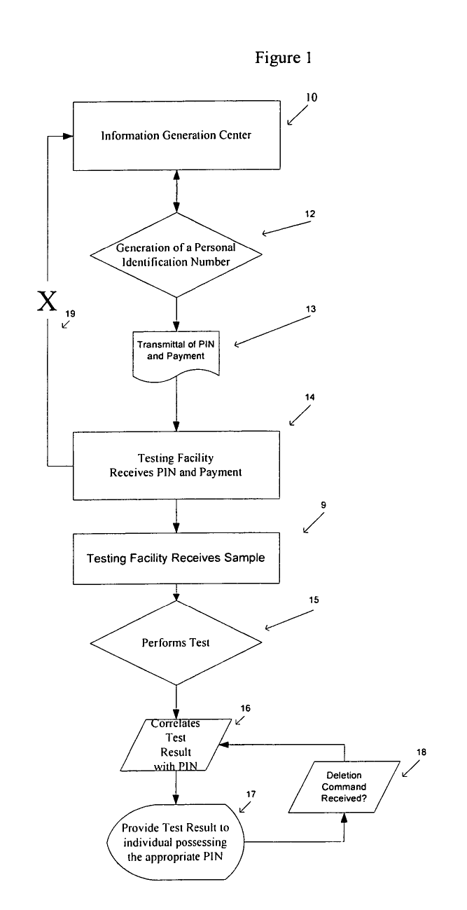

Referring now to FIG. 1, there is shown a flow diagram illustrating the general operation of a system for delivering confidential medical test results to patients in accordance with an embodiment of the invention. The IGC receives a patient request and obtains any necessary personal information and payment from the patient. At this time, the IGC may also provide counseling to the patient where appropriate and if necessary. The information generation center generates a PIN, which is preferably a randomly generated and unique identifier. The PIN may include numbers, alphabetical characters and/or other symbols, so long as the PIN does not provide information regarding the patient's identity. The PIN may include one or more separate character strings, such as a password and a user ID, where appropriate. The PIN may be provided in bar code where appropriate and desirable, for example, on a sample container. The PIN and payment are transmitted 13 to an appropriate testing facility. The testing facility merely receives the payment and PIN 14. The testing facility may, where necessary, communicate with the IGC to clarify or acknowledge receipt of payment and the corresponding PIN. It is understood that the IGC will not divulge any information that could lead to the identification of a patient by the testing facility. Subsequently, a biological sample is transmitted to the testing facility 9, which performs the ordered tests 15 and associates the results with the PIN 16. Following the association between the PIN and the results, the patient is provided a method of accessing the results, which can be accessed with the proper PIN. If desired, the patient may then instruct the testing facility to delete a portion or all of the recorded results 18. The patient may optionally delete a portion or all of the posted results at a later date.

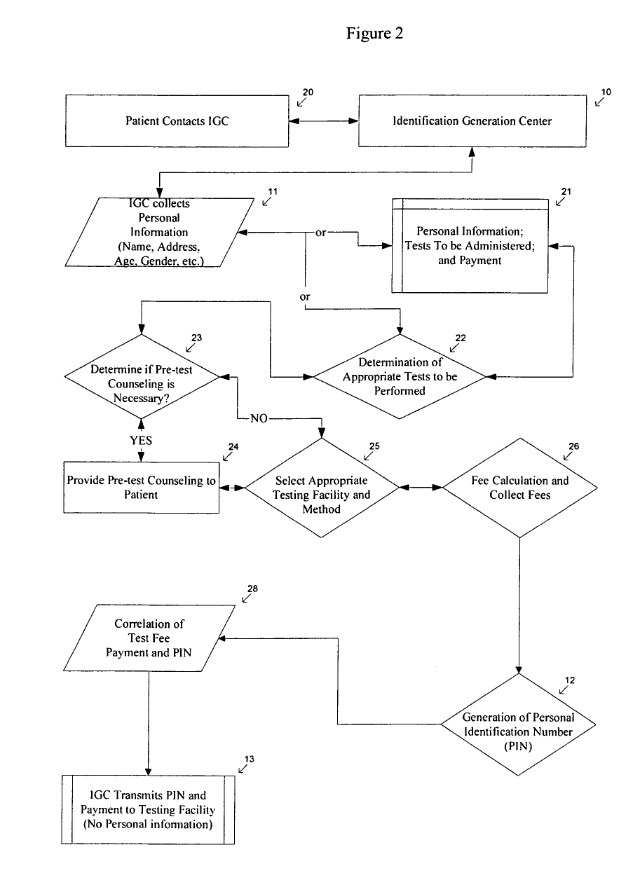

Referring now to FIG. 2, there is shown a flow diagram illustrating the general operation of the IGC. A patient desiring to have a test performed, but wishing to maintain their anonymity, contacts an IGC 20. The IGC will take that personal information 11 necessary to determine or identify the appropriate tests to be conducted. The IGC may store the personal information 21, the nature of the tests to be administered and the payment. The patient and IGC then determine the appropriate tests to be performed 22. Alternatively, the IGC may delete the patients personal information and/or PIN after a specified period of time, after completion of a particular event (e.g., completion of the testing), or other predetermined period of time.

Many of the patients that will benefit most from a system of anonymity are seeking tests that may reveal the presence of terminal illness or serious medical consequences. As a result, the IGC can determine if pre-test counseling is necessary 23. Therefore, pre-testing counseling 24, may be provided by the IGC or the IGC may refer the patient to other sources of counseling. The pre-test counseling can provide an opportunity to assist the patient to cope with the possibility of an adverse test result, referred to herein as a positive result, or gather more information on a particular condition. Pre-testing counseling may also assist the patient in determining what tests should be conducted, the risks and benefits of such tests, the nature of the test (i.e., what a predisposition to cancer means to the patient and how to use the information). Pre-test counseling may affect the patients ultimate decision to have the test performed or may affect the choice of the appropriate test, which may benefit from follow-up counseling. Once the appropriate test(s) are determined 22 and all desired or appropriate pre-test counseling 24 is completed, an appropriate testing facility is selected 25 based on the nature of the tests to be performed. Having determined the desired tests and the testing facility to be used appropriate fees are calculated 26, which includes the fees to be charged by the testing facility 14. Fees for the services provided by the IGC, counselors and/or other services may be included in the fee calculation. The appropriate fees are then paid to the IGC 26. Once the fees are collected or accounted for, the IGC generates a PIN. Preferably, the total fees paid are deposited into an account managed by the IGC, which then reissues the fees necessary to pay the testing facility. Thus, protecting the identification of the patient that would result from the patient paying fees to the testing facility in any form other than cash. Thus, in this embodiment generation of a PIN, which may be performed by a computer that generates a unique random number or character set, automatically connects the payment order to the newly generated PIN 28. The IGC then transmits the PIN and any required payment to the testing facility for completion of the desired testing and to set up an account or database from which the patient may access the test results once the PIN is verified.

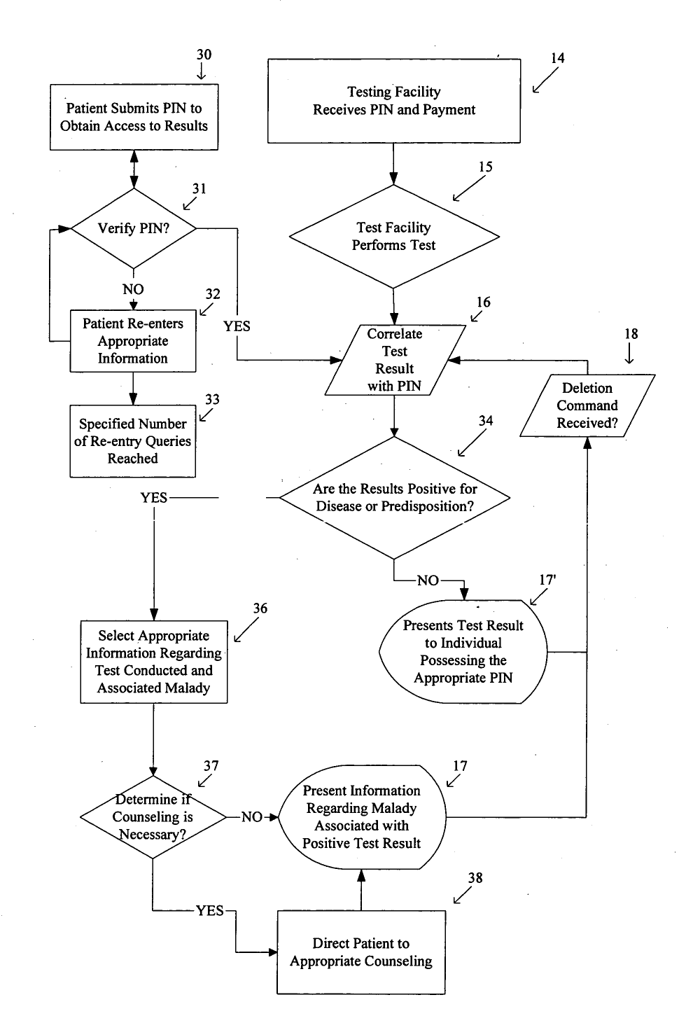

Referring now to FIG. 3, there is shown a flow diagram illustrating the general operation of the testing facility according to a particular embodiment of the invention. The testing facility receives a sample with a PIN for testing 14. The PIN transmitted by the IGC indicates the tests to be performed on the sample. The testing facility then performs the requested tests 14. The tests performed by the testing facility may be any test, for example, the patient may be tested for mutations in breast cancer genes, colon cancer (FAP) genes and HIV infection. Once the tests are performed 15, the test results are associated with or connected to a file or database identified by the patient's PIN 16. Since the invention relates to diseases or predispositions generally having serious ramifications for the patient, one particular embodiment includes identifying positive results 34 (i.e., those results that indicate the presence of a disease state in the patient, indicate a predisposition to a disease state, or the presence of a heritable trait that may be passed to the patient's offspring) and selecting appropriate information 36 to be provided to the patient. The information should correlate with the nature of the test performed, which can effect the information that is appropriate to the results. Furthermore, where desirable and appropriate, a determination may be made as to whether or not to provide counseling to the patient 37. Such counseling may be in addition to any pre-testing counseling provided by the IGC. The test results are then provided to the patient 17 or 17′.

Providing the results to the patient 17 or 17′ requires that the patient present the appropriate PIN and, optionally, other information, wherein the other information does not compromise their identity. For example, the patient may access the information through a web site by entering the PIN 30 and other information (such as, a “User name,” which may alternatively be provided by the IGC as a part of the PIN, access codes identifying the nature of the tests performed, the date the PIN was established or the like). The PIN and other information is then verified 31 by checking the entry against a database having the assigned PINs therein. If the entry does not satisfy the verification requirements the patient may be asked to re-enter the appropriate information. The system may be designed to terminate contact (or disallow access to results) if an incorrect entry is made more than a specified number of times. Upon entry of the correct PIN and other information, the test results 17 or 17′ are shown to the patient. The patient may be prompted with a question asking if the patient would like to delete the record of the results 18. The prompt may allow the patient to delete all of the information or selected aspects of the information.

Alternatively, the patient may be provided the results through a telephone system. The telephone system also requires that the patient present the appropriate PIN and other information, wherein the other information does not compromise the patient's identity. In this embodiment, the patient may access the information by entering the PIN and, optionally, other information (such as, access codes identifying the nature of the tests performed, the date the PIN was established or the like) using the telephone touch-tone buttons. The PIN and other information is verified by checking the entered information against at least one database or reference source having at least the currently assigned PINs therein. If the entry does not satisfy the verification requirements the patient may be asked to re-enter the appropriate information 32. The system may be designed to terminate contact (or disallow access to results) if an incorrect entry is made more than a specified number of times 33, for example, incorrect entry of the PIN three consecutive times may indicate an inappropriate attempt to access information and the call may be terminated. Upon entry of the correct PIN and other information, the test results 17 or 17′ are reported to the patient. The patient may be prompted with a question asking if the patient would like to delete the record of the results 18. The prompt may allow the patient to delete all of the information or selected aspects of the information.

A computer system used with the current invention may include various computer systems coupled over various network connections. For example, a computer network can include a user system, a web server, a file transfer server and a database server. A firewall may separate the user system from the secure file transfer server, which may contain test results. Any suitable systems known in the art may be used in the invention. For example, transaction information may be maintained on a web server by including encrypted information in the URLs. Once encoded, transaction information allows for pages of subsequent visits to incorporate dynamic and pertinent data to the accessing customer at that precise point in time, thereby providing a dynamic environment. A dynamic web environment is one that can produce pertinent, personalized and relevant transactional content based on information provided to the HTTP server and data retrieved from a database, each of which are processed and validated by business applications systems. In one particular embodiment, the encryption algorithms used by the computer program of the secure server are based on publicly available algorithms such as RC4 and MD5. Variations on these cryptographic algorithms may be used or other cryptographic algorithms may be used in accordance with the invention. These and other cryptographic algorithms are known in the art. See, for example, B. Schneier, “Applied Cryptography,” (Jon Wiley & Sons, Inc. 2d ed. 1996), the contents of which are hereby incorporated by reference.

The test results may be screened for those results that are positive for a disease or predisposition of a disease 34. The positive results may then be correlated with the test performed and/or the malady represented by the test 35 and appropriate information correlated with the tests/malady 36. Optionally, a determination may be made regarding the necessity or desirability of providing counseling to the patient 37. A determination that counseling should be provided can trigger the system to provide the appropriate information. For example, the patient may be provided a form of counseling, wherein information is relayed over the phone, either by an automated voice or by a counselor 38 that is provided via telephone to the patient. The patient then receives the test results and information 17. If the patient tests negative for a human malady or predisposition for a malady, the patient may be provided the test results directly 17′.

Tests that may be used in conjunction with the invention are known in the art and include, but are not limited to those described in U.S. Pat. Nos. 6,465,629; 6,448,041; 6,440,699; 6,423,491; 6,417,342; 6,416,961; 6,372,896; 6,342,581; 6,333,403; 6,316,204; 6,312,909; 6,307,019; 6,218,146; 6,180,776; 6,162,897; 6,090,578; 6,599,719; and 6,599,716, the contents of each of which are incorporated by reference herein. Methods of testing that may be used in conjunction with the invention are known in the art and include, but are not limited to those described in U.S. Pat. Nos. 6,458,536; 6,365,350; 6,340,566; 6,297,051; 6,297,041; and 6,599,700; the contents of which are incorporated by reference.

The testing facility may draw or assist in obtaining the sample. However, where the testing facility draws or assists in obtaining the sample, the testing facility should refer to the patient by the assigned PIN only and should not obtain personal information. Thus, a private physician or medical clinic may serve as a part of the testing facility, so long as the patient is unknown to the physician and the physician accepts the PIN as the patient identification without obtaining personal information that may compromise the patient's anonymity. In such a case, the physician may serve to obtain the sample and may submit the sample to a testing laboratory using the PIN. In addition, payment of fees from the IGC may be made to multiple parties that constitute the testing facility. For example, fees and the PIN may be sent to a private physician, a testing laboratory and an independent reporting service. Alternatively, the patient may use a home kit to obtain the sample and submit the sample and PIN to a testing facility, either in person or by mail. Preferably, when the sample and PIN are submitted by mail, a pre-addressed mailing container that lacks a return address is used.

As illustrated herein, the testing facility may comprise one or more independent services that act to receive the PIN and payment, test the sample and provide the results to the patient. Thus, the private physician, testing laboratory and reporting service can constitute a testing facility, as that term is used herein. It will be clear to a person of ordinary skill in the art that any number of service providers may participate as a testing facility, so long as the PIN provides the primary patient identification and the service provider is shielded from the patient's personal information. Thus, a professional genetic counseling center may provide counseling to the patient, so long as the counseling center is shielded from the patient's personal information. In particular, a counseling center may accept the patient and refer to the patient through the use of the PIN.

Likewise, the IGC may be composed of one or more independent entities. For example, an independent client intake facility that produces the PIN 12 may outsource any desired pre-test counseling. In one embodiment, the patient is provided with the option to participate in pre-test counseling either before the generation of the PIN, as illustrated in FIG. 2, or may participate in pre-test counseling after the generation of a PIN, where the counseling is conducted by reference to the PIN and not through private personal information that may compromise the patient's identity.

In another embodiment, the testing facility, upon receipt of the PIN from the IGC, will generate and store the PIN, the tests to be performed, the estimated time for completion of the tests in a status database. The testing laboratory may then indicate a delay or the completion of one or more tests, which will be used to update the status database. The estimated date of completing the testing may be used to provide the patient with information regarding the availability of the test results. For example, the patient may log onto the web site to access the test results and, after entry of the appropriate PIN and other information, be informed that the test results are not currently available, but may be available following a specified date and/or time.

In yet another embodiment, a database is provided, wherein receipt of the PIN and any payment to the testing laboratory, receipt of the PIN and payment to the reporting service, and receipt of the PIN and payment to any counseling provider are stored. The database may be maintained by any aspect of the testing facility. In particular, the database may be provided and maintained by a test-reporting service, thereby providing a central results distribution site that may utilize multiple independent testing laboratories. This is especially beneficial where the tests requested by a patient are diverse and require different testing laboratories and/or different methods of obtaining the sample (e.g., a home use kit for capillary blood and a clinic/physician for arterial blood).

As discussed herein, the present invention may be used in conjunction with a confidential home test system for determining whether an individual patient may be carrying an infection (e.g., HIV virus), have a disease or malady, or a predisposition to a disease state or malady. An individual patient desiring to use the home test system may purchase a test kit at a pharmacy or other retail outlet. The test kit contains a sample (or specimen) container that may be submitted to a testing laboratory. Each test kit purchased by a patient includes a unique multi-digit PIN which may be printed on the sample container. After purchasing a test kit, a patient places an appropriate amount of a sample (e.g., blood, tissue, or other body fluids) into or onto the sample container while at home and then mails the sample container to a testing laboratory for performance of the test. Prior to mailing the sample container to testing laboratory, the patient maintains a record of the PIN printed on his or her sample container. In an alternative embodiment, an IGC may provide or instruct a patient to obtain a home test kit, and may also provide a pre-addressed mailing container and/or a label having a PIN to attach to the sample container. As sample containers are received and processed by the testing laboratory, the testing laboratory may provide digital signals representative of status information (e.g., whether a sample container was received by the lab, the condition of the sample container, the date and time of receipt, whether evaluation of the sample has been delayed, etc.) and result information (e.g., HIV positive, screen positive, HIV negative, error or inconclusive) to a host computer having one or more status databases associated with or based on the PINs on the sample containers received by testing laboratory. The host computer preferably stores test status information received from the testing laboratory in a PIN status database and test result information received from the testing laboratory in a test results database.

After an appropriate period of time following submission of the sample for testing, the patient can place a telephone call to an automated call handler and routing system or may log onto a web site to obtain the testing results and/or to receive counseling. During the course of the telephone call or in logging onto the web site, the patient never reveals his or her identity, but instead receives the test results and counseling by providing the unique PIN to the automated call handler and routing system or web site. Preferably, the reporting service of the testing facility 14 provides a help menu for either the telephone reporting system or the web based reporting system.

In accordance with a another embodiment, an automated web page and routing system facilitates the providing of the test result and counseling information to the patient either via a prepared web page or a series of prepared messages which may be displayed on one or more web pages. Alternatively, the web page may direct the patient to a counselor and/or customer service representatives. Previously prepared messages are may be used by the web site and routing system for informing a patient of test results. Alternatively, in the case of patients that test positive or screen positive for the a malady or predisposition (e.g., HIV virus), or whose test results are inconclusive, the web site and routing system may transfer the patient to live counselors who then inform the patient of their test results. In cases where the test results for the patient are not yet available because the testing laboratory has not completed the test associated with the patient's PIN, the web site may transfer the patient to a recorded message for the patient indicating the date that the patient should log back in to obtain the test results. Alternatively, the patient may be directed to a web page having prepared information relevant to the malady, predisposition, and/or the nature of the test result. For example, the patient may be directed to a comprehensive database of referral organization information including, the names, addresses and phone numbers of individuals and organizations that can provide additional information or counseling relating to the malady or predisposition, such as those provided by the U.S. Centers for Disease Control.

In order to promptly display status information or test results, a host computer is connected to a current status and results database. The database(s) maintain test result and status information for each sample or test by associating all information pertaining to the test with a PIN. The testing laboratory and host computer are preferably coupled by a dedicated modem line, LAN or internet connection, and, on a regular basis, the testing laboratory provides the host computer with current status and result information for all sample containers received and results for all tests performed.

The reporting system may initially provide a series of automated messages to patients who have tested negative, to repeat patients who have previously accessed information regarding a positive test result and who were previous flagged to receive counseling or announcements, and to patients for whom test results are not available. A reporting system employing a web page to display test results preferably uses a secure server and is encrypted to display test results to a patient who has correctly verified the PIN. In this manner, verification is used to route the patient to a secure site. Preferably, the patient may then select a properly identified button to access the results and is offered the choice of deleting some or all of the results from the database.

In addition, counselors that have conveyed positive test result information to a patient or a patient having positive test results may be flagged to receive “counseling announcements” in the event the patient logs in or calls again. If the patient is flagged for “counseling announcements,” this flagging information can be stored with the patient's PIN in the PIN database and, in the event the patient logs in or calls again, the patient can be transferred to the appropriate resource.

In the event that the patient has exceeded the maximum number of times for receiving the test results, a message may be provided by the reporting system indicating the last date and time that test result information was provided to the patient. A patient may be granted a predetermined number of accessions to the reporting web site or reporting telephone service before the patient's access is terminated. This may be accomplished, for example, by recording the number of accessions and terminating the validity of the PIN after a specified number of accessions is granted. Alternatively, the PIN may have an expiration date. However, where a PIN is assigned an expiration date, the expiration date may be extended.

From the foregoing, it will be observed that numerous modifications and variations can be effected without departing from the true spirit and scope of the novel concept of the present invention. It will be appreciated that the present disclosure is intended as an exemplification of the invention, and is not intended to limit the invention to the specific embodiment illustrated. The disclosure is intended to cover by the appended claims all such modifications as fall within the scope of the claims.

1. A method for anonymously testing a patient, the method comprising:
assigning a unique numerical identifier to a patient and at least one test for a medical condition, wherein the unique numerical identifier identifies the patient and the at least one test to be performed for the patient;
transmitting the unique numerical identifier to a testing facility;
receiving a biological sample from the patient at the testing facility, wherein the biological sample is identified by the unique numerical identifier;
performing the at least one test on the biological sample to detects the medical condition and determining a test result, wherein the at least one test is performed by the testing facility;
correlating the test result from the at least one test with the unique numerical identifier;
receiving a request for the test result from the patient, wherein the patient is identified by the unique numerical identifier; and
providing the test result to the patient, thereby protecting the anonymity of the patient.

2. The method according to claim 1, wherein the biological sample is obtained at the testing facility and the testing facility uses the unique numerical identifier to identify the patient.

3. The method according to claim 1, wherein receiving the request from the patient is conducted through an internet site.

4. The method according to claim 3, wherein providing the test result to the patient is conducted through the internet site.

5. The method according to claim 1, further comprising providing pre-test counseling prior to assigning the unique numerical identifier.

6. The method according to claim 1, wherein the medical condition is selected from the group consisting of HIV, Herpes, Breast cancer, Colon cancer, Hemophilia, Duchenne's muscular dystrophy, Becker's muscular dystrophy, Adrenoleukodystrophy, Phenylketonuria, Long QT syndrome, Beta thalassemia, Cystic Fibrosis, Fragile X, Huntington's disease, Hurler syndrome, Lesch Nyhan syndrome, Neurofibromatosis, Spinal Muscular Atrophy, Von Hippel Lindau and Alzheimers.

7. The method according to claim 1, further comprising:
determining if the patient has a positive test result for a human malady or predisposition to a human malady; and
providing information regarding the human malady or the predisposition to a human malady to the patient prior to providing the test result.

8. The method according to claim 7 wherein the information is provided by a counselor.

9. The method according to claim 8, wherein the counselor provides the test result.

10. The method according to claim 1, wherein the biological sample is selected from the group consisting of mucosal fluid, blood, urine and feces.

11. The method according to claim 1, wherein the testing facility draws the biological sample from the patient.

12. A method for anonymously testing a patient, the method comprising:
assigning a patient a unique numerical identifier and a test for a medical condition, wherein the unique numerical identifier identifies the patient and the test to be performed for the patient;
transmitting the unique numerical identifier to a testing facility;
receiving a biological sample from the patient at the testing facility, wherein the biological sample is identified by the unique numerical identifier;
performing the test on the biological sample to detect the medical condition and determining a test result;
correlating the test result from the test with the unique numerical identifier;
receiving a request for the test result from the patient through an internet site, wherein the patient is identified by the unique numerical identifier;
verifying the unique numerical identifier against a database of valid unique numerical identifiers after receiving the request; and
providing the test result to the patient over the internet site if the unique numerical identifier is valid, thereby protecting the anonymity of the patient.

13. The method of claim 12, further comprising transferring the patient's internet contact to a secure server, prior to providing the test result.

14. The method of claim 12, wherein the unique numerical identifier is assigned by an entity separate from the testing facility.

15. The method of claim 12, further comprising:
determining if the patient has a positive test result for a human malady or predisposition to a human malady; and
providing information regarding the human malady or the predisposition to a human malady to the patient prior to providing the test result.

16. The method according to claim 15, wherein the information comprises textual information.

17. The method according to claim 15, further comprising:
querying the patient to determine if the patient desires counseling; and
providing the patient with counseling, wherein a counselor provides the test result.

18. The method of claim 17, further comprising:
querying the patient regarding desired online counseling; and
providing an instant messaging account number, an instant messaging account, and instructions for using the instant messaging account, wherein the patient has indicated a desire for online counseling, and connecting the counselor by the instant messaging account to the patient, thereby providing additional information to the patient.

19. The method according to claim 17, wherein providing the patient with counseling is conducted through e-mail of a secure web page.

20. The method according to claim 12, wherein the biological sample is selected from the group consisting of mucosal fluid, blood, urine and feces.
